(19) United States
(12) Patent Application Publication
(10) Pub. No.: US 2022/0133360 A1
(43) Pub. Date: May 5, 2022

(54) Spinal Fixation Assembly

(71) Applicant: Lenkbar LLC, Naples, FL (US)

(72) Inventors: Erik Papenfuss, Naples, FL (US); Robert Zasada, Naples, FL (US); Terry Clawson, Naples, FL (US)

(73) Assignee: Lenkbar LLC, Naples, FL (US)

(21) Appl. No.: 17/434,032

(22) PCT Filed: Feb. 27, 2020
(86) PCT No.: PCT/US2020/020007

§ 371 Date: Aug. 26, 2021

Related U.S. Application Data
(60) Provisional application No. 62/810,979, filed on Feb. 27, 2019.

(51) Int. Cl. A61B 17/70

(52) U.S. Cl. CPC A61B 17/7032; A61B 17/7037; A61B 17/7091

(57) ABSTRACT

A fixation assembly includes a pedicle screw having a screw head, an elongate tulip head inserted over the pedicle screw, and a swivel rocker inserted into the tulip head. The swivel rocker has an axial through passage extending therethrough, an arcuate concave distal face being contoured to match the screw head, and a saddle shaped proximal face. A rod assembly is seated on the saddle shaped proximal face. A retainer ring secures the tulip head around the screw head. A method of implanting the assembly is also provided.

### BACKGROUND OF THE INVENTION

### Field of the Invention

The present invention relates to a spinal fixation assembly.

### Description of the Related Art

Implants are often inserted into adjacent vertebrae to lock the vertebrae together, such as in instances when the disc between the vertebrae has been removed due to injury or other problem with the disc. Because of the location of the implants in vertebrae and proximate to the spinal cord, implant procedures tend to be relatively long, involved processes. Once an implant is implanted, it is typically not desirable to remove the implant due to the delicate location of the implant as well as a loss of purchase and time after taking out the implant and its associated screws and re-inserting.

It would be beneficial to provide a fixation assembly that readily allows for the attachment and removal of fixation constructs with minimal effort.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is an assembly that is used to flexibly join adjacent vertebrae. The assembly includes a pedicle screw, a tulip head that is adjustable mounted on the head of the pedicle screw. The tulip head is used to support a rod or other construct. A retainer ring around the tulip head allows for adjustment of the tulip head on the pedicle screw and also for locking the tulip head to the pedicle screw.

In an alternative embodiment, a method of implanting the assembly is also provided.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a perspective view of a fixation assembly with rod according to an exemplary embodiment of the present invention;

FIG. 2 is an exploded perspective view of the fixation assembly with rod of FIG. 1;

FIG. 3 is a side elevational view, in section of the fixation assembly of FIG. 2;

FIG. 4 is a side elevational view of a rod according to an exemplary embodiment of the present invention;

FIG. 5 is a sectional view of the rod of FIG. 4, taken along lines 5-5 of FIG. 4;

FIG. 6 is a perspective view of an installer tool according to an exemplary embodiment of the present invention;

FIG. 7 is an exploded view of the installer tool of FIG. 6;

FIG. 8 is a side elevational view, in section of the distal end of the installer tool of FIG. 6, taken along lines 8-8 of FIG. 6; and

FIG. 9 is a perspective view of the sectioned distal tip of the installer tool of FIG. 8.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. As used herein, the term “distal” is defined as a location farther from an implanting clinician and the term “proximal” is defined as a location closer to the implanting clinician.

The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

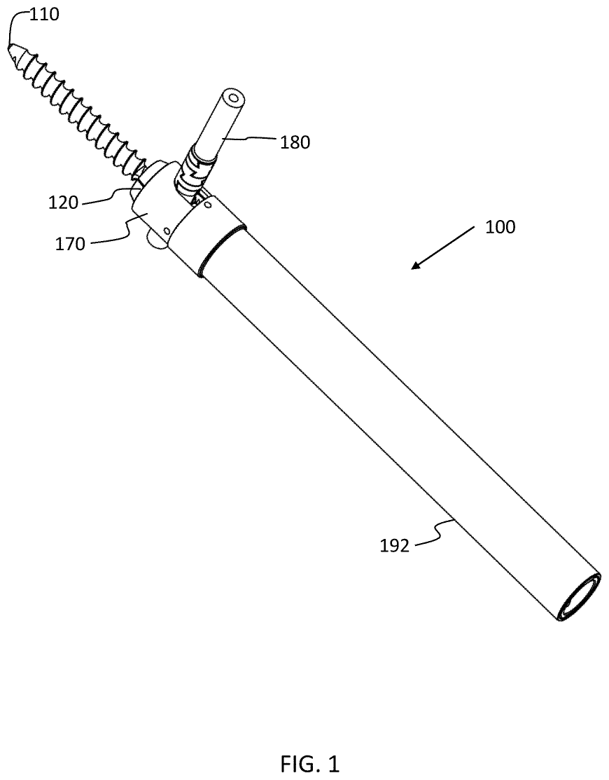

Referring to FIGS. 1-4, a fixation assembly 100 (“assembly 100”) according to an exemplary embodiment of the present invention is shown. Assembly 100 is used to join adjacent vertebrae (not shown), yet still allow some movement and flexibility of one vertebra with respect to the connected vertebra.

Assembly 100 includes a pedicle screw 110 having a distal insertion tip 112, a proximal screw head 114 located distally from insertion tip 112, and a threaded body 116 extending along a screw axis 111 between insertion tip 112 and head 114.

Pedicle screw 110 can be a known polyaxial screw that allows and can be cannulated or solid. Additionally, screw head 114 can be lacerated, patterned, or otherwise scored to form a relatively rough surface.

Head 114 is generally circular in shape and includes a receiver 118 that is adapted to receive an insertion end of torqueing device (not shown). Receiver 118 can include a polygonal cross-section such as, for example, hexagonal, Torx®, or other shape, to allow an insertion device (not shown) to be inserted into receiver 118 and rotate screw 110 into a vertebra.

Assembly 100 further includes an elongate tulip head 120 that can be inserted over head 114. Tulip head 120 is generally cylindrically shaped with a through passage 122 extending therethrough along a tulip head axis 121. Tulip head 120 includes an interior body portion 124 having a conical distal end 123 that is beveled to form a cone-shaped receiver for head 114. Side walls 126 in body portion 124 can be lacerated, patterned, or otherwise scored to form a relatively rough surface. When tulip head 120 is inserted over head 114, the rough surface of side walls 126 and the rough surface of head 114 provide a relatively larger frictional relationship between side walls 126 and head 114 to further prevent rotation of tulip head 120 with respect to screw 110 after implantation. An exterior of distal end 123 also includes a distal band 125 that extends around a periphery of distal end 123.

Tulip head 120 further includes a pair of diametrically opposed arcuately shaped proximal fingers 128 extending proximally from body portion 124. A space 129 formed between each finger 128 is sized to allow the insertion of a rod assembly 180 having an axis 181 that extends generally orthogonally to tulip head axis 121. The distal end 130 of space 129 is generally arcuate, with a radius of curvature matching the radius of curvature of rod assembly 180 so that rod assembly 180 can be seated in the distal end 130.

A through opening 131 is provided in each side wall 126 along each finger 128. Through openings 131 are spaced 180 degrees apart from each other. Each through opening 131 is sized to receive a pin 134. A proximal end portion 136 of each finger 128 includes an internal thread 138. A proximal band 139 extends around the periphery of proximal end portion 136. Proximal band 139 extends outwardly the same distance as distal band 125.

A score line 137 extends peripherally around each finger 128, about half way along internal thread 138. Score lines 137 allow proximal ends of fingers 128 to be snapped off of tulip head 120 after assembly 100 is implanted.

A blind slot 142 is formed in the exterior of each finger 128 along proximal end portion 136. Blind slot 142 is generally oblong in shape and extends parallel to tulip head axis 121.

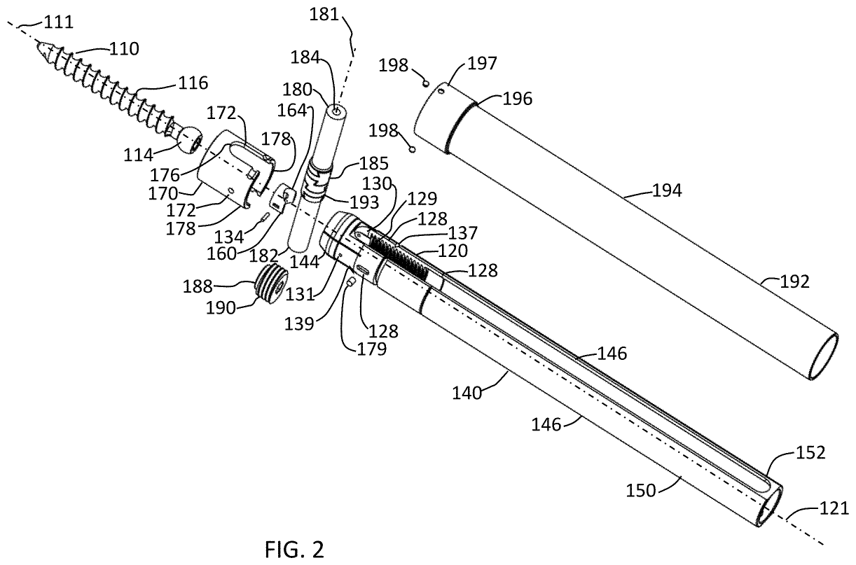

Tulip head 120 also includes a plurality of longitudinal through slots 144 (only one through slot 144 shown in FIG. 2) extending from the distal end of body portion 124 and partially along the length of each finger 128. Slot 144 allows body portion 124 to expand outwardly when body portion 124 is being fitted over screw head 114.

An unthreaded extension 146 extends proximally from each finger 128. A weld 148 connects each extension 146 to its respective finger 128. Extensions 146 are welded to fingers 128 for manufacturing purposes and, with different manufacturing techniques, weld 148 may be eliminated. Extension 146 can be different lengths, depending on clinician desires.

A proximal end 150 of tulip head 120 is connected at a connection 152 such that extensions 146 are connected to each other. Connection 152 prevents fingers 128 from splaying open and away from each other. Additionally, connection 152 aids in sliding an installation tube 192 over tulip head 120.

A swivel rocker 160 is inserted into tulip head 120 and advanced distally of internal threads 138 of fingers 128. Swivel rocker 160 has an axial through passage 161 extending therethrough. Swivel rocker 160 includes an arcuate concave distal face 162 that is contoured to match the generally circular contour of screw head 114 and a saddle shaped proximal face 164 that is contoured to match the cylindrical contour of rod assembly 180 so that rod assembly 180 can be seated on saddle shaped proximal face 164. Two blind slots 166 (only one blind slot 166 is shown) are formed 180 degrees apart from each other in the exterior of swivel rocker 160 along proximal end portion 136. Blind slot 166 is generally rectangular in shape and extends parallel to tulip head axis 121.

Each blind slot 164 is aligned with a through opening 131 in tulip head and pin 134 is inserted through the through each opening 134 and into each slot 164. Pin 134 is sufficiently long to extend into both blind slots 164 and through opening 134 without extending radially outside of tulip head 120. Blind slot 164 has a length that is longer than the diameter of pin 134 so that swivel rocker 160 can travel inside tulip head 120 and allow swivel rocker 160 to tightly engage screw head 114 when assembly is gully installed and tightened or to “relax” away from screw head 114 and allow tulip head 120 to rotate about screw head 114 when aligning tulip head 120 during implantation.

A retainer ring 170 is used to secure body portion 124 of tulip head 120 around head 114 so that head 114 cannot inadvertently back out of body portion 124. Retainer ring 170 is slid distally over body portion 124 after head 114 is inserted into body portion 124. Retainer ring 170 is sized to just fit over distal band 125 and proximal band 139 of tulip head 120. When retainer ring 170 is slid over tulip head 120, a clearance of about 0.005 inches is provided between the exterior surface of tulip head 120 between bands 125, 139 and retainer ring 170.

Retainer ring 170 includes a pair of diametrically spaced arcuate legs 172 that extend proximally from a distal ring portion 174. A longitudinally extending space 176 is formed between the legs 172. Space 176 is approximately the same width as space 129 in tulip head 120.

A through opening 177 is formed in retainer ring such that, when retainer ring 170 is slid over tulip head 120, through opening 177 is aligned with blind slot 142 in tulip head. A pin 179 is inserted into through opening 177 and into blind slot 142. Pin 179 is smaller than the length of blind slot 142 such that retainer ring 170 can slide distally and proximally relative tulip head 120, with pin 179 sliding in blind slot 142 and restrict the amount of travel of retainer ring 170 relative to tulip head 120. In an exemplary embodiment, between about 2 mm and about 3 mm of travel is provided for pin 179 to slide within slot 142. This travel allows a distal end of retainer ring 170 to slide proximally of band 125 and allow side walls 126 of tulip head 120 to splay open along slots 144 for insertion or removal of head 114 into or out of tulip head 120.

A proximal portion of each leg 172 includes a pair of peripherally extending slots 178 that is each in communication with an adjacent space 176 such that a slot 178 extends peripherally in opposing directions from space 176.

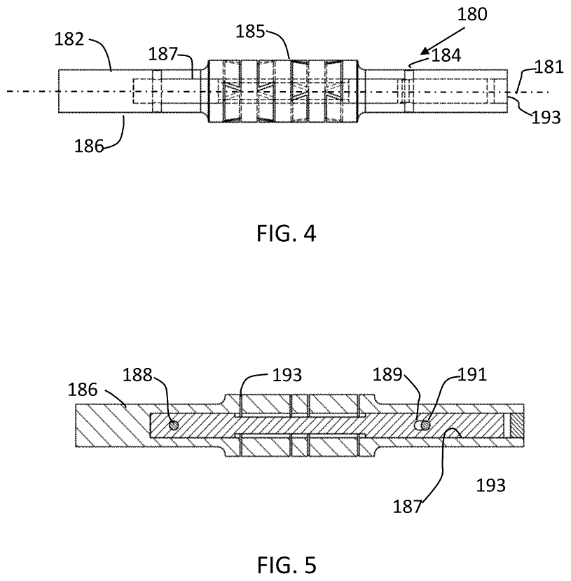

Referring now to FIGS. 2 and 4, rod assembly 180 is a dynamic stabilization rod that includes a first rod portion 182 on one side of rod assembly 180, a second rod portion 184 on an opposing side of rod assembly 180, and a flexible portion 185 between first rod portion 182 and second rod portion 184. An exemplary rod assembly 180 is disclosed in U.S. Pat. No. 8,366,559, which is owned by the Assignee of this invention and which is incorporated herein by reference in its entirety. Flexible portion 185 allows rod assembly 180 to flex and bend in a restrained manner, providing a person in whom assembly 100 is implanted with flexibility over standard, prior art rod implants. In an exemplary embodiment, flexible portion 185 can have a larger diameter than first rod portion 182 and second rod portion 184 or, alternatively, flexible portion 185 can be the same diameter as first rod portion 182 and second rod portion 184.

Rod 180 can be constructed from a generally hollow outer body 186 constructed from 17-4 stainless steel, with a nitinol inner rod 187 inserted therein. Inner rod 187 can be fixedly attached to first rod portion 182, such as by a pin 188 extending through both first rod portion 182 and inner rod 187. Optionally, inner rod 187 can be omitted. While stainless steel and nitinol are used in rod 180, those skilled in the art will recognize that other materials, such as titanium, can be used.

Inner rod 187 at second rod portion 184 includes a slot 189 into which a pin 191 is inserted. Slot 189 allows inner rod 187 to “float” within outer body 186, but still be restrained by pin 191 as rod 180 flexes. In an exemplary embodiment, slot 189 allows for about 1 millimeter of travel. A cap 193 at the end of second rod portion 184 seals inner rod 187 inside outer body 186.

Flexible portion 185 includes a plurality of slots 193 formed therein. Slots 193 allow rod 180 to bend. Slots 193 can be cut in any direction. As shown in FIG. 2, slots 193 are cut in a dovetail pattern, resulting in more surface area, which gives more strength on push, pull and torque and provide the opportunity to expand, contract (length wise) bend, twist and be used as a shock absorber.

In an exemplary embodiment, rod 180 can bend about 6 degrees away from rod axis 181. The width of slots 193 can vary to adjust the degree of flexion of rod 180. Inner rod 187 can be narrower along flexible portion 185. Optionally, instead of flexible rod 180, a rigid rod, such as just first rod portion 182 can also be used.

First rod portion 180 is inserted distally into tulip head 120 such that rod portion 182 is advanced through space 129 and into saddle shaped proximal face 164. Second rod portion 184 is inserted into a second tulip head 120 in a second assembly 100 so that the two assemblies 100 are each implanted into adjacent vertebrae (not shown), with rod 180 spanning the gap between the vertebrae.

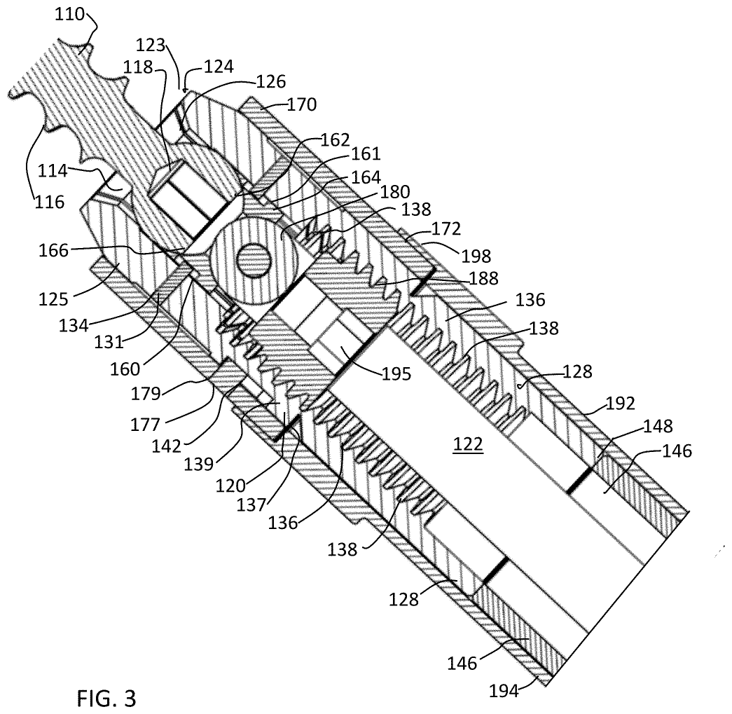

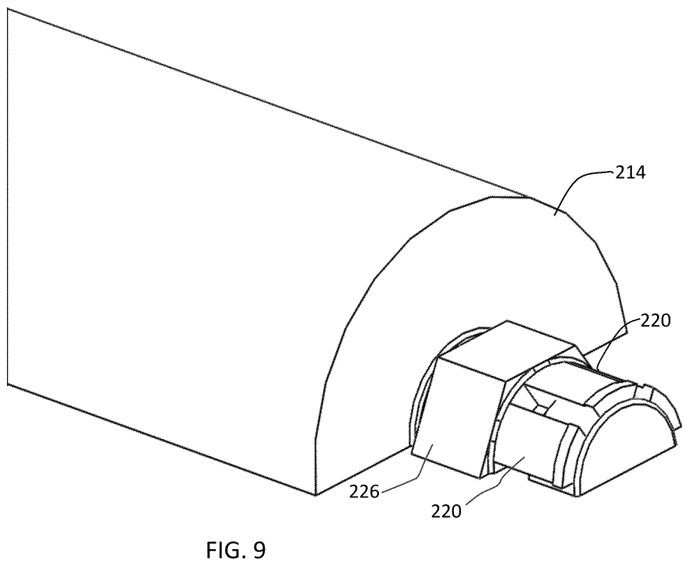

Referring back to FIGS. 2 and 3, a lockdown nut 188 has external threads 190 that are sized to engage threads 138 of tulip head 120. Lockdown nut 188 is inserted into tulip head 120 and threaded distally until lockdown nut 188 engages first rod portion 182 and secures first rod portion 182 inside tulip head 120. Lockdown nut 188 includes a receiver 195 that is adapted to receive hexagonal support 226 of grabber rod 210 (shown in FIG. 9). While a hexagonal receiver 195 and support 226 are shown, those skilled in the art will recognize that other shapes, such as, for example, octagonal, Torx®, or other shape, to allow support 226 to be inserted into receiver 195 and rotate lockdown nut 188 into tulip head 120. In an exemplary embodiment, lockdown nut 188 is initially attached to insertion rod assembly 200 and inserted into tulip head 120 after tulip head 120 is attached to screw 110.

An installation tube 192 has a body 194 that is sized to be inserted over proximal end of tulip head 120 with minimal side clearance between body 194 and tulip head 120 so that installation tube 192 can be slid distally along tulip head 120. Installation tube 192 includes a distal portion 196 having a larger diameter than body 194 so that distal portion 196 can slide over retainer ring 170.

Distal portion 196 includes a pair of diametrically opposed through openings 197 (only one opening 197 shown in FIG. 2) that receive pins 198. Each pin 198 extends into a respective space 176 in retainer ring 170. Installation tube 192 can be rotated either clockwise or counterclockwise relative to tulip head 120 such that pins 198 extend into a slot 178 to restrict assembly 100 from rotating while lockdown nut 188 is being threaded into tulip head 120. A lip 198 connecting body 194 with distal portion 196 engages proximal end of retainer ring 170 when installation tube 192 is fully inserted over tulip head 120.

In an exemplary embodiment, crew 110, tulip head 120, swivel rocker 160, retainer ring 170, and lockdown nut 188 can be constructed from titanium. Installation tube 192 can be constructed from 17-4 stainless steel. Those skilled in the art, however, will recognize that other materials can be used.

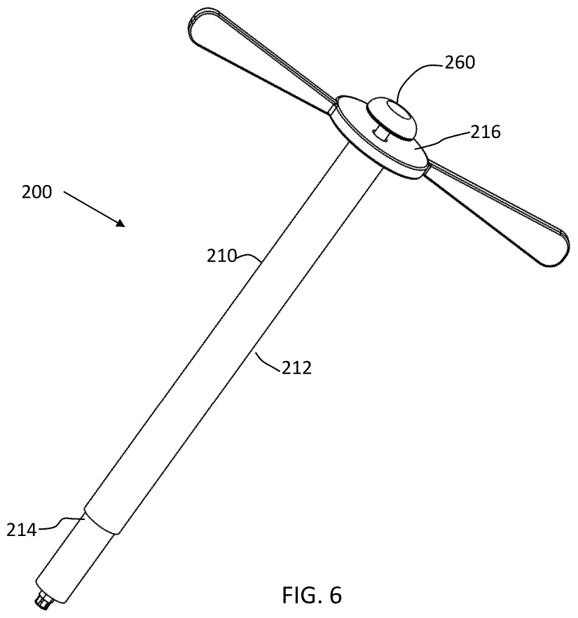

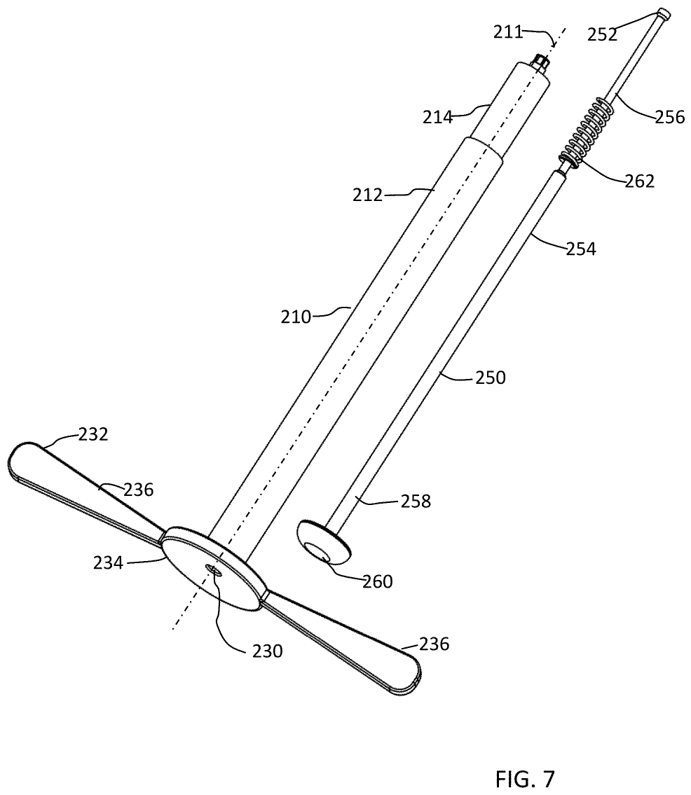

An insertion rod assembly 200 (“rod assembly 200”) is shown in FIGS. 6-9. Rod assembly 200 is used to attach and remove tulip head 120 over screw head 114.

Rod assembly 200 includes a generally hollow outer installer body 210 and an inner grabber rod 250 that translates within installer body 210. Installer body 210 has an elongate tubular body 212 having a distal end 214 and a proximal end 216 with a passage 218 having a longitudinal axis 211 extending therethrough.

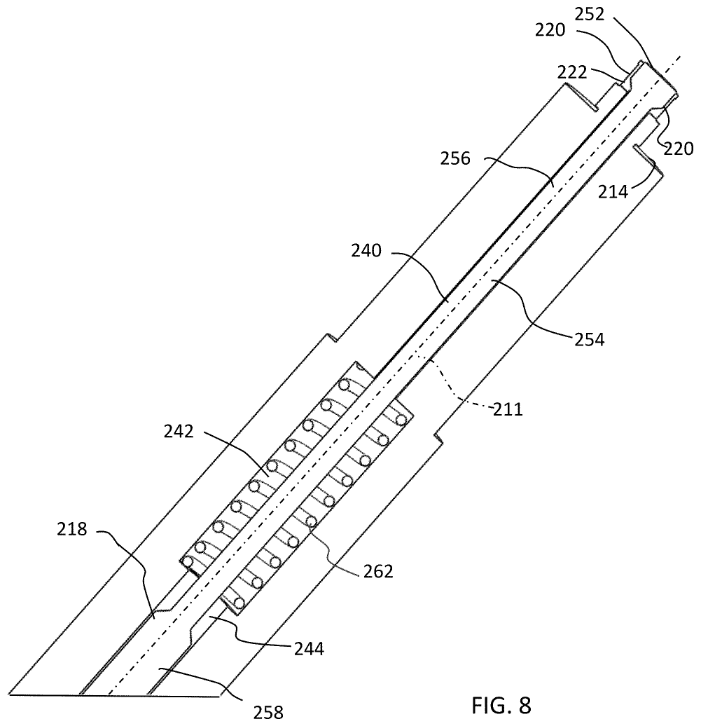

Distal end 214 includes a plurality of arcuate fingers 220 spaced around longitudinal axis 211 in a circle. FIG. 8 shows a concave landing 222. A hexagonal support 226 supports fingers 220. Hexagonal support 226 is sized and shaped to fit into receiver 195 in lockdown nut 188.

Proximal end 216 has a through opening 230 that is aligned with longitudinal axis 211. A gripping portion 232 extends generally orthogonally to longitudinal axis 211. In an exemplary embodiment, gripping portion 232 includes a circular base 234 with a plurality of wings 236 extending radially therefrom. In an exemplary embodiment, two wings 236 are shown, although those skilled in the art will recognize that more than two wings 236 can be used.

Passage 218 has a distal portion 240 having a first diameter, a central portion 242 having a second diameter, larger than the first diameter, and a proximal portion 244 having a third diameter, smaller than the second diameter. The third diameter can be the same size or a different size than the first diameter.

The differing diameter sizes effectively form ledges at the boundaries between distal portion 240 and central portion 242 and between central portion 242 and proximal portion 244.

Grabber rod 250 includes a distal plug 252 that rests inside wings 236. Plug 252 is connected to a shaft 254 that extends through passage 218. Shaft 254 has a distal end 256 having a first diameter to fit within first and second diameters of passage 218 and a proximal end 258 having a second diameter, larger than the first diameter, to fit within third diameter of passage 218.

A generally button shaped pusher 260 is attached to proximal end 258 and extends exteriorly of installer body 210. A tension spring 262 is located around shaft 254 inside passage 218 at central portion 242 of passage, abutting the ledges formed by the different diameters of passage 218. Tension spring 262 biases proximal end of shaft 254 in a proximal direction so that plug 252 is biased to be seated within wings 236.

In an exemplary embodiment installer body 210 and grabber rod are constructed from 17-4 stainless steel and tension spring 262 is constructed from a spring material.

To insert assembly 100, screw 110 is initially screwed into a desired location. Retaining ring 170 is loosened from tulip head 120 and slid proximally, loosening body portion 124 of tulip head so that head 114 can be inserted into passage 122 in body portion 124 of tulip head 120. After head 114 is inserted into passage 122, the generally circular shape of head 114 allows head 114 to swivel about inside passage 122 to allow an infinite variation between screw axis 111 and tulip head axis 121.

Installation tube 192 can be inserted over tulip head 120 and rotated so that pins 198 extend into a respective space 176 in retainer ring 170 to prevent assembly 100 from rotating. First rod portion 182 can be inserted into tulip head 120 through spaces 129 in tulip head 120. Rod assembly 180 is manipulated to a desired position relative to screw 110. Retaining ring 170 is slid distally so that distal band 125 is compressed by retaining ring 170 against body portion 124, securing tulip head 120 to screw head 114.

Insertion rod assembly 200, with lockdown nut 188 attached to wings 236 of installer body 210, is inserted into installation tube 192 so lockdown nut 188 engages threads 138. Lockdown nut 188 is fully threaded distally along threads 138 to engage first rod portion 182 and secure rod assembly 180 to assembly 100.

Lockdown nut 188 engages first rod portion 182 and urges first rod portion 182 distally against saddle shaped proximal face 164 of swivel rocker 160. Swivel rocker 160 in turn is pushed distally so that concave distal face 162 of swivel rocker 160 is urged against screw head 114, further securing tulip head 120 to screw 110.

Pusher 260 is advanced distally to push plug 252 away from wings 236 and to push wings 236 away from lockdown nut 188. With rod assembly 200 separated from lockdown nut 188, insertion rod assembly 200 is removed from installation tube 192 and installation tube 192 is rotated to move pins 198 from their respective space 176 in retainer ring 170 and removed proximally from tulip head 120. Retainer ring 170 is then advanced distally so that side walls 126 are compressed against screw head 114, further securing tulip head 120 to screw 110.

It will be further understood that various changes in the details, materials, and arrangements of the parts which have been described and illustrated in order to explain the nature of this invention may be made by those skilled in the art without departing from the scope of the invention as expressed in the following claims.

1. A fixation assembly comprising:
a pedicle screw having a distal insertion tip, a proximal screw head located distally from insertion tip, and a threaded body extending along a screw axis between the insertion tip and the screw head;
an elongate tulip head inserted over the screw head, the tulip head comprising:
a through passage extending therethrough along a tulip head axis;
an interior body portion having a conical distal end being beveled to form a cone-shaped receiver for the screw head;
a pair of diametrically opposed arcuately shaped proximal fingers extending proximally from the body portion such that a space formed between each finger is sized to allow the insertion of a rod assembly having a rod axis extending generally orthogonally to the tulip head axis;
a plurality of longitudinal through slots extend from the distal end of body portion and partially along the length of each finger, such that the slots allows the body portion to expand outwardly when the body portion is being fitted over the screw head;
a swivel rocker is inserted into the tulip head, the swivel rocker having:
an axial through passage extending therethrough;
an arcuate concave distal face being contoured to match the screw head; and
a saddle shaped proximal face;
a rod assembly seated on the saddle shaped proximal face; and
a retainer ring securing the body portion of the tulip head around the screw head.

2. The fixation assembly according to claim 1, wherein the head includes a receiver that is adapted to receive an insertion end of a torqueing device.

3. The fixation assembly according to claim 1, wherein a distal end of the space is generally arcuate, with a radius of curvature matching the radius of curvature of the rod assembly such that the rod assembly can be seated in the distal end.

4. The fixation assembly according to claim 1, wherein a through opening is provided in each side wall along each finger.

5. (canceled)

6. The fixation assembly according to claim 4, wherein each through opening is sized to receive a pin.

7. The fixation assembly according to claim 4, wherein each finger comprises a proximal end portion and wherein the proximal end portion of each finger includes an internal thread.

8. The fixation assembly according to claim 7, further comprising a proximal band extending around a periphery of the proximal end portion.

9. The fixation assembly according to claim 8, further comprising a blind slot formed in an exterior of each finger along the proximal end portion.

10. The fixation assembly according to claim 9, wherein the blind slot is generally oblong in shape and extends parallel to the tulip head axis.

11. The fixation assembly according to claim 1, wherein an unthreaded extension extends proximally from each finger.

12. The fixation assembly according to claim 11, further comprising two blind slots formed 180 degrees apart from each other in swivel rocker along a proximal end portion of each finger.

13. The fixation assembly according to claim 13, further comprising a pin, wherein each blind slot is aligned with a through opening in the tulip head and wherein the pin is inserted through the through each through opening 134 and into each blind slot.

14. The fixation assembly according to claim 13, wherein the pin is sufficiently long to extend into both blind slots and through the through opening without extending radially outside of the tulip head.

15. The fixation assembly according to claim 1, wherein the retainer ring includes a pair of diametrically spaced arcuate legs extending proximally from a distal ring portion of the retainer ring.

16. The fixation assembly according to claim 15, wherein a proximal portion of each leg includes a pair of peripherally extending slots that is each in communication with an adjacent space such that a slot extends peripherally in opposing directions from space.

17. The fixation assembly according to claim 1, further comprising an insertion rod assembly having:
a generally hollow outer installer body; and
an inner grabber rod that translates within installer body.

18. The fixation assembly according to claim 17, wherein the installer body has an elongate tubular body having a distal end and a proximal end with a body passage having a body longitudinal axis extending therethrough.

19. The fixation assembly according to claim 18, wherein the distal end includes a plurality of arcuate fingers spaced around the body longitudinal axis in a circle.

20. The fixation assembly according to claim 19, further comprising a hexagonal support supporting the arcuate fingers.

21. The fixation assembly according to claim 17, wherein the proximal end of the elongate tubular body has a body through opening that is aligned with the body longitudinal axis.

22-29. (canceled)
